(19) United States
(12) Patent Application Publication
(10) Pub. No.: US 2006/0014677 A1
(43) Pub. Date: Jan. 19, 2006

(54) Method for maximizing efficacy and predicting and minimizing toxicity of calcineurin inhibitor compounds

(75) Inventor: Patrick Rogers Mayo, Edmonton (CA)

Correspondence Address: ISOTECHNIKA INC., 17301 NORTH PERIMETER DRIVE, SUITE 100, SCOTTSDALE, AZ, 85255, US

(73) Assignee: Isotechnika International Inc.

(21) Appl. No.: 11/181,580

(22) Filed: Jul. 13, 2005

Related U.S. Application Data
(60) Provisional application No. 60/633,165, filed on Dec. 3, 2004.
(60) Provisional application No. 60/589,365, filed on Jul. 19, 2004.

(51) Int. Cl. A61K 38/13; A61K 31/4745

(52) U.S. Cl. 514/11; 514/183; 514/291

(57) ABSTRACT

The invention provides methods for predicting toxicity related to calcineurin inhibition therapy by measuring the peak concentration of drug and the trough concentration of the drug, calculating a peak-trough fluctuation, and comparing this peak-trough fluctuation to known values to predict if the patient will exhibit calcineurin-inhibition therapy-related toxicity. The invention also provides methods for monitoring drug levels to ensure that a patient receiving calcineurin inhibition therapy remains within a therapeutic window which maximizes the efficacy and minimizes the toxicity of the calcineurin inhibitor. The invention also provides dosage methods which maximize the peak concentration, minimize the trough concentration, and maximize the fluctuation between peak and trough concentration of calcineurin inhibitors, to maximize the efficacy of the calcineurin inhibition therapy, and minimize the risk of developing calcineurin-inhibition therapy-realted toxicity. This dose regimen, which may be a once-daily dose regimen, maximizes efficacy associated with peak concentrations of drug and minimizes toxicity by maximizing the peak-trough fluctuation, a measurement determined to be associated with toxicity. Calcineurin inhibitors useful for these methods include members of the cyclosporin family of compounds, including cyclosporin A and ISA247, FK506, pimecrolimus and ascomycin.

### FIELD OF THE INVENTION

The invention provides methods for dosing and monitoring patients receiving calcineurin inhibitor therapy.

### References

U.S. Pat. No. 4,108,985.
U.S. Pat. No. 4,210,581.
U.S. Pat. No. 4,220,641.
U.S. Pat. No. 4,288,431.
U.S. Pat. No. 4,384,996.
U.S. Pat. No. 4,396,542.
U.S. Pat. No. 4,554,351.
U.S. Pat. No. 4,771,122.
U.S. Pat. No. 4,894,366.
U.S. Pat. No. 4,916,138.
U.S. Pat. No. 4,929,611.
U.S. Pat. No. 5,229,116.
U.S. Pat. No. 5,284,826.
U.S. Pat. No. 5,478,577.
U.S. Pat. No. 5,525,590.
U.S. Pat. No. 5,672,360.
U.S. Pat. No. 5,891,474.
U.S. Pat. No. 6,159,501.
U.S. Pat. No. 6,280,770.
U.S. Pat. No. 6,329,153.
U.S. Pat. No. 6,432,445.
U.S. Pat. No. 6,451,776.
U.S. Pat. No. 6,465,016.
U.S. Pat. No. 6,468,967.
U.S. Pat. No. 6,475,519.
U.S. Pat. No. 6,551,619
U.S. Pat. No. 6,558,702.
U.S. Pat. No. 6,589,549
U.S. Pat. No. 6,599,527.
U.S. Pat. No. 6,565,504.
U.S. Pat. No. 6,605,593.
U.S. Pat. No. 6,610,735.
U.S. Pat. No. 6,613,739.
U.S. Patent Publication No. 2002/0142946.
U.S. Patent Publication No. 2003/0109426.
U.S. Patent Publication No. 2003/0166515.
U.S. Patent Publication No. 2004/1057768.
U.S. Patent Publication No. 2004/0110666.
U.S. Patent Publication No. 2003/0186855.
International Patent Publication No. WO 86/02080.
International Patent Publication No. WO 99/18120.
International Patent Publication No. WO 03/017947.
International Patent Publication No. WO 03/033010.
European Patent Publications No. 0 034 567.
European Patent Publications No. 0 056 782.
Baral, et.al. Pharmacokinetic Dosing of Aminoglycosides: A Controlled Trial. Am. J. Med. 2003; 114:194-198.
Bennett, W. M., “The nephrotoxicity of new and old immunosuppressive drugs,” Renal Failure, Vol. 20, pp. 687-90 (1998).
Cole, E. et.al. Clinical Benefits of Neoral C2 Monitoring in the Long-Term Management of Renal Transplant Recipients. Transplantation 2003: 75:2086-2090.
Freitag et al., Abstract of the 3^{rd} International Congress on Immunosuppression, Dec. 9, 2004.
Fruman DA. Klee CB. Bierer BE. Burakoff SJ. Calcineurin phosphatase activity in Tlymphocytes is inhibited by FK 506 and cyclosporin A. Proceedings of the National Academy of Sciences of the United States of America. 1992; 89(9):3686-90.
Granelli-Piperno A, Andrus L, Steinman RM. LLymphokine and nonlymphokine mRNA levels in stimulated human cells: kinetics, mitogen requirements, and effects of cyclosporin A. J Exp Med 1986; 163:922.
Ismail, R. et.al. Therapeutic Drug Monitoring of Gentamicin: a 6-year Follow-Up Audit. J. Clin. Pharm. And Therapeutics, 1997. 22:21-25.
Klecka, William R. Discriminant Analysis. Quantitative Applications in the Social Sciences Series, No. 19. Thousand Oaks, Calif.: Sage Publications 1980.
Kobel et al., Europ. J. Applied Microbiology and Biotechnology 14, 237-240 (1982).
Morales, J. et.al. A Simple Method to Calculate Cyclosporine Dosage to Obtain a Target C2 Drug Level. Therapeutic Drug Monitoring, 2003. 25:389-392.
Traber et al., Helv. Chim. Actam 60, 1247-1255 (1977).
Traber et al., Helv. Chim. Acta, 65, 1655-1667 (1982).
Triggs, E. and Charles, B. Pharmacokinetics and Therapeutic Drug Monitoring of Gentamicin in the Elderly. Clin. Pharmacokinet 1999. 37(4): 331-341.
Uijtendaal, E. V., et.al. Once-Daily Versus Multiple-Daily Gentamicin in Infants and Children Therapeutic Drug Monitoring, 2001; 23:506-513.
von Wartburg et al., Progress in Allergy, 38, 28-45 (1986). (Sketris et al., 1995).
(Valantine and Schroeder, 1995).
Wenger, Agnew. Transpl. Proc. 15, Suppl. 1:2230 (1983).
Wenger Agnew. Chem. Int. Ed., 24, 77 (19855).
Wenger, Agnew. Progress in the Chemistry of Organic Natural Products 50, 123 (1986).

### BACKGROUND

Immunosuppression can be accomplished by inhibiting the activity of a ubiquitous enzyme, calcineurin. Calcineurin inhibition is a delicate therapy, however. Too much calcineurin inhibition can result in unacceptable side effects. Too little calcineurin inhibition, for a therapy such as prevention of transplant rejection, can result in unacceptable and life-threatening organ rejection. Calcineurin inhibitors include members of the cyclosporin family, including cyclosporin A, analogs and derivatives of cyclosporin A such as cyclosporins B through Z, ISA247, FK506 ascomycin and pimecrolimus.

Cyclosporin A is a potent immunosuppressive agent that has been demonstrated to suppress humoral immunity and cell-mediated immune reactions such as allograft rejection, delayed hypersensitivity, experimental allergic encephalomyelitis, Freund's adjuvant arthritis and graft vs. host disease. It is used for the prophylaxis of organ rejection subsequent to organ transplantation; for treatment of rheumatoid arthritis; for the treatment of psoriasis; and for the treatment of other autoimmune diseases, including type I diabetes, Crohn's disease, lupus, and the like.

FK506, also known as tacrolimus and sold as Prograf® was described in U.S. Pat. Nos. 4,894,366, 4,916,138 and 4,929,611 and is available from Fujisawa. First described in 1987, FK506 is a derivative of a soil fungus. FK506 is used for immunosuppression, including immunosuppression following organ transplant. It has very similar immunosuppressive properties to cyclosporine, but is 10 to 100 times more potent on a per gram basis. Related compounds, pimecrolimus, sold in a topical formulation as Elidel® by Novartis, and ascomycin, are also calcineurin inhibitors.

There are numerous adverse effects associated with calcineurin inhibition therapy. Cyclosporine A therapy has been associated with adverse effects including nephrotoxicity, hepatotoxicity, cataractogenesis, hirsutism, parathesis, and gingival hyperplasia to name a few (Sketris et al., 1995). Of these, nephrotoxicity is one of the more serious, dose-related adverse effects resulting from cyclosporine A administration. It has been disclosed that immediate-release cyclosporine A drug products (e.g., Neoral® and Sandimmune®) can cause nephrotoxicities and other toxic side effects due to their rapid release and the absorption of high blood concentrations of the drug. Cyclosporin A is also commercially available in a soft gelatin capsule form in 25, 50 and 100 mg doses as Genfrafg from Abbott. Side effects of FK506 treatment include kidney damage, seizures, tremors, high blood pressure, diabetes, high blood potassium, headache, insomnia, confusion, seizures, neuropathy, and gout.

Cyclosporins are a class of cyclic polypeptides, consisting of eleven amino acids, that are produced as secondary metabolites by the fungus species Tolypocladium inflatum Gams. Examples of this class of drug are described in The Merck Index, Thirteenth Edition, page 480 which is herein incorporated by reference. They have been observed to reversibly inhibit immunocompetent lymphocytes, particularly T-lymphocytes, in the G0 or G1 phase of the cell cycle. Cyclosporine derivatives have also been observed to reversibly inhibit the production and release of lymphokines (Granelli-Piperno et al., 1986). Although a number of cyclosporine derivatives are known, cyclosporine A is the most widely used. The immunosuppressive effects of cyclosporin A is related to the inhibition of T-cell mediated activation events. This suppression is accomplished by the binding of cyclosporine to the ubiquitous intracellular protein, cyclophilin. This complex, in turn, inhibits the calcium- and calmodulin-dependent serine-threonine phosphatase activity of the enzyme calcineurin. Inhibition of calcineurin prevents the activation of transcription factors such as NFAT_{p/c} and NF-κB, which are necessary for the induction of the cytokine genes (IL-2, IFN-γ, IL-4, and GM-CSF) during T-cell activation. FK506 inhibits calcineurin similarly, except that FK506 acts through a different immunophilin protein, dubbed FK binding protein. Cyclosporine also inhibits lymphokine production by T-helper cells in vitro and arrests the development of mature CD8 and CD4 cells in the thymus (Granelli-Piperno et al., 1986). Other in vitro properties of cyclosporine include the inhibition of IL-2 producing T-lymphocytes and cytotoxic T-lymphocytes, inhibition of IL-2 released by activated T-cells, inhibition of resting T-lymphocytes in response to alloantigen and exogenous lymphokine, inhibition of IL-1 production, and inhibition of mitogen activation of IL-2 producing T-lymphocytes (Granelli-Piperno et al., 1986).

Since the original discovery of cyclosporin, a wide variety of naturally occurring cyclosporins have been isolated and identified and many further non-natural cyclosporins have been prepared by total- or semi-synthetic means or by the application of modified culture techniques. The class comprised by the cyclosporins is thus now substantial and includes, for example, the naturally occurring cyclosporins A through Z [c.f. Traber et al. (1977); Traber et al. (1982); Kobel et al. (1982); and von Wartburg et al. (1986)], as well as various non-natural cyclosporin derivatives and artificial or synthetic cyclosporins including the dihydro- and iso-cyclosporins; derivatized cyclosporins (e.g., in which the 3′-O-atom of the -MeBmt-residue is acylated or a further substituent is introduced at the α-carbon atom of the sarcosyl residue at the 3-position); cyclosporins in which the -MeBmt-residue is present in isomeric form (e.g., in which the configuration across positions 6′ and 7′ of the -MeBmt-residue is cis rather than trans); and cyclosporins wherein variant amino acids are incorporated at specific positions within the peptide sequence employing, e.g., the total synthetic method for the production of cyclosporins developed by R. Wenger—see e.g. Traber et al. (1977), Traber et al. (1982) and Kobel et al. (1982); U.S. Pat. Nos. 4,108,985, 4,210,581, 4,220,641, 4,288,431, 4,554,351 and 4,396,542; European Patent Publications Nos. 0 034 567 and 0 056 782; International Patent Publication No. WO 86/02080; Wenger (1983); Wenger (1985); and Wenger (1986). Cyclosporin A analogues and derivatives containing modified amino acids in the 1-position are reported by Rich et al. (1986). Immunosuppressive, anti-inflammatory, and anti-parasitic cyclosporin A analogues are described in U.S. Pat. Nos. 4,384,996; 4,771,122; 5,284,826; and 5,525,590, all assigned to Sandoz. Additional cyclosporin analogs and derivatives have been disclosed in U.S. Patent Publications No. 2002/0142946, 2003/0109426, 2003/0166515, WO 03/017947, WO 03/033010, 2004/1057768,2004/01106662003/0186855 and U.S. Pat. No. 6,551,619. FK506, another macrocyclic calcineurin inhibitor, has been disclosed in US Pats. No. 4,894,366, 4,916,138 and 4,929,611. Additional cyclosporin analogs and derivatives are disclosed in WO 99/18120, U.S. Pat. Nos. 6,605,593, 6,613,739 assigned to Isotechnika. The terms Ciclosporin, ciclosporin, cyclosporine, and Cyclosporine are interchangeable and refer to the class of cyclosporin compounds which include cyclosporin A and ISA247.

Calcineurin inhibitors are difficult to dose. These drugs exhibit considerable variability in blood concentration of drug between patients, between pharmaceutical agents, and between formulations. In addition, these drugs exhibit significant side effects. It is preferable to dose these drugs so that their immunosuppressive effects are sufficient to create the desired pharmaceutical effect, while minimizing the side effects associated with calcineurin inhibition therapy. There is thus a need for an improved method for dosing calcineurin inhibitor drugs such as cyclosporine, cyclosporine analogs and FK506, that offers greater treatment efficacy and reduced toxicity associated with these agents. In addition, there is a need for a method for predicting when a patient will experience toxic side effects of these therapies.

### SUMMARY

Embodiments of the present invention provide methods for predicting toxicity related to calcineurin inhibition therapy by measuring the peak concentration of drug and the trough concentration of the drug, calculating a peak-trough fluctuation, and comparing this peak-trough fluctuation to known values to predict if the patient will exhibit calcineurin-inhibition therapy-related toxicity. Embodiments also provide methods for monitoring drug levels to ensure that a patient receiving calcineurin inhibition therapy remains within a therapeutic window which maximizes the efficacy and minimizes the toxicity of the calcineurin inhibitor. In additional embodiments dosage methods are provided which maximize the peak concentration, minimize the trough concentration, and maximize the fluctuation between peak and trough concentration of calcineurin inhibitors, to maximize the efficacy of the calcineurin inhibition therapy, and minimize the risk of developing calcineurin-inhibition therapy-realted toxicity. This dose regimen, which may be a once-daily dose regimen, may maximizes efficacy associated with peak concentrations of drug and may minimize toxicity by maximizing the peak-trough fluctuation, a measurement determined to be associated with toxicity. Calcineurin inhibitors useful for these methods include members of the cyclosporin family of compounds, including cyclosporin A, and analogs, derivatives, amides, esters, isomers and prodrugs thereof, ISA247 and analogs, derivatives, amides, esters, isomers and prodrugs thereof and FK506 and analogs, derivatives, amides, esters, prodrugs and related compounds including pimecrolimus and ascomycin, and their analogs, derivatives, amides, esters, prodrugs and related compounds.

An embodiment of this invention provides a method for maximizing the fluctuation between the peak concentration of calcineurin inhibitors as a class, including cyclosporin and cyclosporin-related compounds such as ISA247 and the trough concentration of calcineurin inhibitors, where maximizing the peak concentration of the calcineurin inhibitor is associated with maximizing the efficacy of the compound in inhibiting calcineurin activity and where minimizing the trough concentration of the calcineurin inhibitor minimizes toxicity and side-effects of the therapy, including renal toxicity.

Another embodiment of his invention relates to a method for predicting calcineurin toxicity based on a patient's peak-trough fluctuation. The less peak-trough fluctuation a patient exhibits, the greater the probability that the patient will suffer side effects associated with calcineurin inhibition therapy, specifically renal toxicity as measured by increasing levels of serum creatinine.

In another embodiment, this invention provides a once-daily dosing regimen for calcineurin inhibitors such as cyclosporin and cyclosporin-related compounds such as ISA247 which maximizes peak concentration and maximizes efficacy, minimizes trough concentration and minimizes toxicity, and maximizes the peak-trough fluctuation, a predictor for cylosporin-related renal toxicity.

An embodiment of the present invention provides a method for administering a calcineurin inhibitor to a patient in need of calcineurin inhibition therapy which optimizes efficacy of the calcineurin inhibitor and minimizes calcineurin inhibitor-related toxicity comprising maximizing the fluctuation between a peak calcineurin inhibitor concentration and a trough calcineurin inhibitor concentration. An embodiment provides that the calcineurin inhibitor is cyclosporine A, cyclosporine A derivatives, ISA247 and FK506, pimecrolimus, and ascomycin. Another embodiment provides that the calcineurin inhibitor is administered once daily. Further, an embodiment provides that the method for administering the calcineurin inhibitor minimizes the trough concentration or maximizes the amount of time that the patient is at the trough concentration.

In an additional embodiment, the invention provides a method for administering a calcineurin inhibitor where the calcineurin inhibitor is administered once daily and where the once daily dose maximizes peak concentration of the calcineurin inhibitor and minimizes trough concentration of the calcineurin inhibitor. In an additional embodiment, the once daily dose method maximizes peak-trough fluctuation. In an additional embodiment, the calcineurin inhibitor is cyclosporine A, cyclosporine A derivatives, analogs, amides, esters, isomers and prodrugs thereof, ISA247 and analogs, derivatives, amides, esters, isomers and prodrugs thereof and FK506 and analogs, derivatives, amides, esters, prodrugs and related compounds including pimecrolimus and ascomycin, and their analogs, derivatives, amides, esters, prodrugs and related compounds.

In a still further embodiment, the present invention provides a method for monitoring a patient receiving calcineurin inhibitor therapy comprising: (1) measuring the patient's peak concentration of a calcineurin inhibitor; and, (2) measuring the patient's trough concentration of a calcineurin inhibitor. An additional embodiment provides that the monitoring method further provides (3) calculating a peak-trough fluctuation; and, (4) using the calculated peak-trough fluctuation as a marker to monitor for the development of calcineurin-inhibitor therapy-related toxicity in the patient wherein a smaller peak-trough fluctuation indicates a greater probability that the patient will suffer calcineurin inhibition therapy-related toxicity. Another embodiment provides that the calcineurin inhibitor is cyclosporine A, ISA247, FK506 pimecrolimus or ascomycin and analogs, derivatives, amides, esters, isomers, prodrugs and related compounds.

In an additional embodiment, the invention provides that when the calculated peak-trough fluctuation is below 350%, toxicity is predicted.

Another embodiment of the present invention provides a method for monitoring a patient receiving calcineurin inhibition therapy to predict calcineurin inhibition therapy-related toxicity in a patient comprising: (1) measuring the patient's peak concentration of a calcineurin inhibitor; and (2) measuring the patient's trough concentration of a calcineurin inhibitor. In a further embodiment, the calcineurin inhibitor may be cyclosporine A, ISA247, FK506, pimecrolimus or ascomycin and their analogs, derivatives, amides, esters, isomers, prodrugs and related compounds.

In an additional embodiment, the invention provides a method for predicting calcineurin inhibition therapy-related toxicity in a patient comprising: (1) measuring the patient's peak concentration of a calcineurin inhibitor; (2) measuring the patient's trough concentration of a calcineurin inhibitor; (3) calculating a peak-trough fluctuation; and, (4) using the calculated peak-trough fluctuation to predict toxicity in the patient wherein a smaller peak-trough fluctuation indicates a greater probability that the patient will suffer calcineurin inhibition therapy-related toxicity.

An embodiment further provides that when the calculated peak-trough fluctuation is below 350%, toxicity is predicted. Further, an embodiment provides that the calcineurin inhibitor may be cyclosporine A, ISA247, FK506, pimecrolimus or ascomycin and their analogs, derivatives, amides, esters, isomers, prodrugs and related compounds.

### BRIEF DESCRIPTION OF THE FIGURES

FIG. 1 shows an Emax model showing the correlation between % calcineurin inhibition and Emax (%).

FIG. 2 shows a Psoriasis Area and Severity Index vs. Trough Concentration of a cyclosporin-related compound, ISA247.

FIGS. 3a and 3b show correlations between trough concentration and Emax (%) for ISA247 and Cyclosporin A (Neoral).

FIGS. 4a and 4b show Peak (C2) correlations with Maximum Calcineurin Inhibition (Emax) for ISA247 and Cyclosporin A (Neoral).

FIG. 5 shows concentration vs. time profiles of drug concentration for patients treated with ISA247.

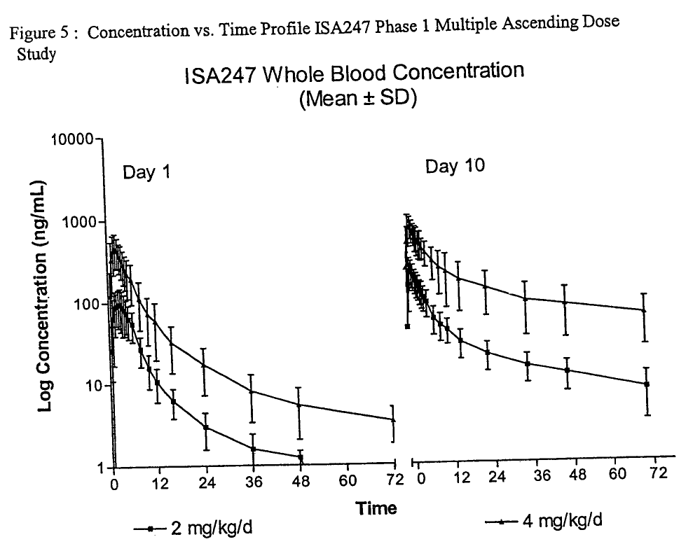

FIGS. 6a and 6b show compartmental (phase) analysis for ISA247 concentration time curve.

FIG. 7 shows a concentration vs. time curve for once-daily dosing of ISA247.

FIG. 8 illustrates the effect of sustained release on efficacy and toxicity of treatment with cyclosporin or cyclosporin-related compounds.

### DETAILED DESCRIPTION OF THE EMBODIMENTS

Patients receiving calcineurin inhibitors such as cyclosporin and FK506 are carefully monitored to ensure that their therapeutic levels are sufficient to create the desired pharmaceutical effect, and to ensure that they are not experiencing side effects associated with calcineurin inhibition therapy. These patients are routinely tested to determine the concentration of drug in their blood. Toxicities associated with cyclosporin A and FK506 are severe, especially renal toxicity in renal transplant patients, and physicians keep careful watch on their patients to be sure that their drug doses are not too high and reaching toxic levels. However, if drug levels are too low, the consequences of insufficient therapeutic effect can be severe. A transplant patient taking an immunosuppressive agent may experience life-threatening organ rejection if the therapy is not within the effective therapeutic window.

To complicate therapeutic dose monitoring in patients receiving calcineurin inhibition therapy, cyclosporin A is available in several formulations which have different bioavailability profiles. And, there is a known intra-patient variability in bioavailability of cyclosporin A. These factors make patient monitoring a difficult and dangerous business.

In addition to individual variability between patients in the bioavailability of these agents, there is significant individual variability between patients with regard to toxic side effects. Some patients are simply more likely to experience toxic side effects than others. There is a need for methods for predicting when a patient will experience toxic side effects of these therapies.

The structure of cyclosporin A is illustrated in formula (1):

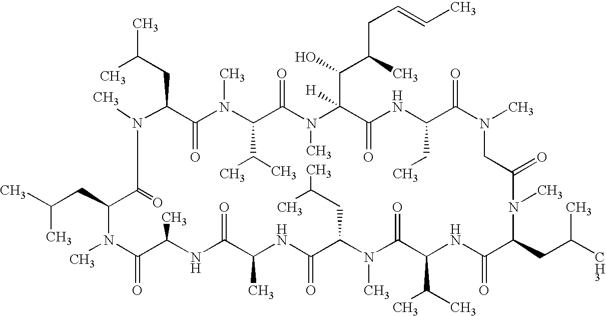

The structure of ISA247 is illustrated in Formula (2A) and (2B):

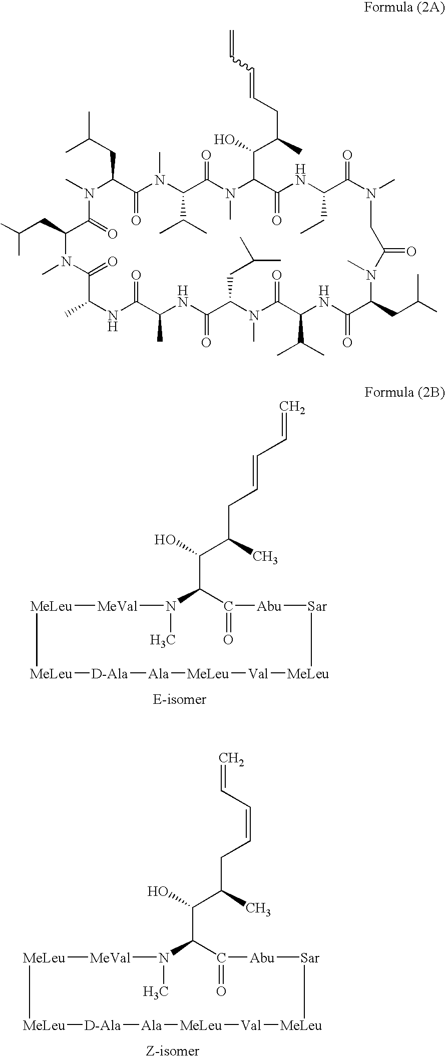

The structure of FK506 is illustrated in Formula (3):

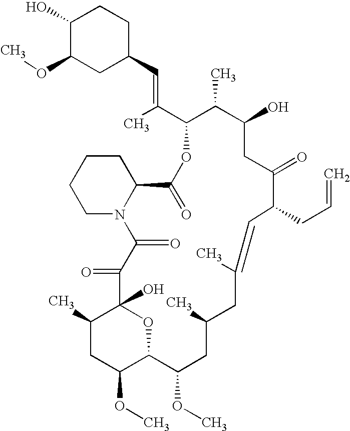

The structure of pimecrolimus is illustrated in Formula (3):

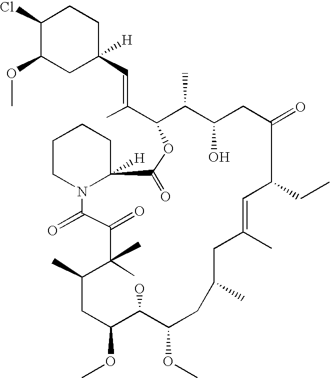

The structure of ascomycin is illustrated in Formula (4):

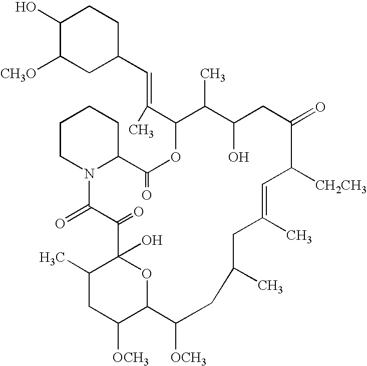

Bioavailability of any drug, including calcineurin inhibitors such as cyclosporin A, ISA247, FK506, pimecrolimus and ascomycin, can vary depending on the patient. In the case of cyclosporin A, Neoral® dosage forms can carry up to about 100 mg/mL of cyclosporine and the dosage form can be relatively large. The absolute bioavailability of cyclosporine administered as Sandimmune® is highly variable and dependent on the patient or the patient population. In liver transplant patients, for example, absolute bioavailability is estimated to be less than 10% while absolute bioavailability may be as high as 89% in some renal transplant patients for which cyclosporine therapy is indicated.

AUC, a measurement of the amount of drug in the body over a period of time, is a measurement that is routinely taken on patients receiving cyclosporin A therapy. AUC may be highly variable with different formulations of cyclosporin. In studies of renal transplant, rheumatoid arthritis and psoriasis patients, the mean cyclosporine AUC is known to be approximately 20% to 50% greater and the peak blood cyclosporine concentration (Cmax) approximately 40% to 106% greater following administration of Neoral® compared to following administration of Sandimmune®. The dose normalized AUC in de novo liver transplant patients administered Neoral® 28 days after transplantation is known to be 50% greater and Cmax 90% greater than in those patients administered Sandimmune®. In another indication for cyclosporine therapy, AUC and Cmax are also increased (Neoral® relative to Sandimmune®) in heart transplant patients.

Following oral administration of currently available dosage forms of cyclosporin A, absorption of cyclosporin A is known to be incomplete. The extent of absorption of cyclosporin A is dependent on the individual patient, the patient population, and the formulation. The relationship between administered dose and exposure (area under the concentration versus time curve, AUC) is linear within the therapeutic dose range. Intersubject variability of cyclosporine exposure (determined by comparing AUC) when Neoral.RTM or Sandimmune.RTM is administered ranges from approximately 20% to 50% in renal transplant patients. This intersubject variability contributes to the need for individualization of the dosing regimen for optimal therapy. Intrasubject variability of AUC in renal transplant recipients (% CV) is known to be 9%-21% for Neoral.RTM and 19%-26% for Sandimmune.RTM. when intrasubject variability of trough concentrations (% CV) is 17%-30% for Neoral.RTM. and 16%-38% for Sandimmune.RTM.

Currently, dosing of the calcineurin inhibitors cyclosporine and FK506 (tacrolimus) are approved in the United States to be administered orally twice daily. Cyclosporine and FK560 are available as a regular release soft-gelatin capsule and tablet, respectively. Traditionally whole blood level trough (C0) concentrations have been utilized to adjust drug dosage. Recently, Novartis has recommended the use of two-hour (C2) monitoring for dosage adjustment of cyclosporin, however this practice has not be universally adopted in all transplant centers. No attempt has been made to optimize peak trough fluctuation of either drug, merely to obtain a C0 or C2 level deemed to be appropriate to prevent rejection. To date no once daily formulations of cyclosporine or FK506 are approved for use in the United States or Canada.

It has been postulated that the peak concentrations of the drug are associated with toxic side effects (Bennett, 1998). The exact mechanism by which cyclosporine A causes renal injury is not known; however, it is proposed that an increase in the levels of vasoconstrictive substances in the kidney leads to the vasoconstriction of the afferent glomerular arterioles. This can result in renal ischemia, a decrease in glomerular filtration rate and, over the long term, interstitial fibrosis. When the dose is reduced or another immunosuppressive agent is substituted in response to renal toxicity, renal function improves (Valantine and Schroeder, 1995).

With this kind of intrapatient variability and differences in bioavailability between formulations, determining an appropriate dose for a particular patient is difficult for these agents. Predicting toxicity has also been difficult. AUC, as determined by monitoring drug concentration between C0 and C4, has been used to adjust dosage for individual patients. Attempts have also been made to determine an appropriate dose for an individual patient based on a single drug concentration measurement taken 2 hours after a dose of the drug, at C2 (Morales, et al., 2003). C2 cyclosporin measurements have been reported to correlate more strongly with AUC (r2≧0.8) and therefore are a better reflection of systemic exposure. Adjusting cyclosporine drug doses to C2 levels has been shown to result in a decrease in dose with an improvement in renal function. C2 has not been utilized as a marker for single daily dosing.

A cyclosporin derivative, ISA247 (also known as ISA_{TX}247 or ISA) has been disclosed in WO 99/18120, and U.S. Pat. No. 6,605,593, and U.S. Pat. No. 6,613,739. The structure of ISA247 (also known as ISA_{TX}247 or ISA) illustrated in Formulas (2A) and (2B). ISA247 exists in two isomeric forms, as shown in Formula (2B).

ISA247 has been studied as a mixture of the isomeric forms of the drug. The isomeric mixture may range from approximately 50:50 E and Z isomer to an essentially pure E isomer formulation, where the E isomer is present at 85%, 90%, or greater than 90%. The trans form (the E isomer) of ISA247 has been shown in x-ray crystallographic studies (Freitag et al., Abstract of the 3rd International Congress on Immunosuppression, Dec. 9, 2004) to fit more efficiently into the active site of the cyclophilin molecule than the cis form (the Z isomer). ISA247 has been shown to be a more effective calcineurin inhibitor while exhibiting less toxicity compared to cyclosporin A.

Other pharmaceutical agents exhibit significant side effects. For example, Gentamicin is an aminoglycoside antibiotic that is important in the treatment of Gram-negative bacterial infection, but exhibits serious nephrotoxicity side effects. Studies of Gentamicin pharmacodynamics indicate that maximizing the peak concentration (Cmax) of Gentamicin while lowering the trough concentration (C0) may reduce renal toxicity while retaining antimicrobial effectiveness (Bartal, et.al., 2003, Ismail et al., 1997, Triggs and Charles, 1999, Uijtendaal, et al., 2001). Therefore, once-daily dosing of gentamicin is suggested to provide a high peak level and a low trough level (Uijtendaal, et al., 2001).

Daptomycin, another antibiotic agent which exhibits skeletal muscle toxicity, has also been shown to be more effective and less toxic when dosed in long dosing intervals of 24 hours or greater. This long dosing interval allows for higher peak concentrations, related to daptomycin effectiveness, while the long dosing interval results is hypothesized to result in reduced toxicity (U.S. Pat. No. 6,468,967).

Long dosing intervals can equate with low trough levels of drug. It may be that these low trough levels of these drugs which exhibit toxic side effects allow the body to recover from the toxic effects of the drugs. During long trough periods, the body may be able to rest and heal, reducing the damage which may occur as a result of exposure to the pharmaceutical agent.

Calcineurin inhibitors are difficult to dose because these drugs exhibit considerable variability in blood concentration of drug between patients and between formulations. In addition, these drugs exhibit significant side effects. There is a need for an improved method for dosing calcineurin inhibitor drugs such as cyclosporine, cyclosporine analogs such as ISA247, and FK506 and related compounds such as pimecrolimus and ascomycin, that offers greater treatment efficacy and reduces the toxicity associated with these agents. In addition, there is a need for a method for predicting when a patient will experience toxic side effects of these therapies.

Two separate pharmacokinetic analyses (Study A and Study B) of clinical trial data relating to ISA247 show that the efficacy of calcineurin inhibitor drugs is related to the peak concentrations of the drugs and not trough concentrations of the drugs (in Study A), and that nephrotoxicity is associated with the fluctuation between the peak and trough concentrations of the drug (in Study B). Study B also shows that patients who experience nephrotoxicity exhibit much less fluctuation between peak (Cmax) and trough (C0) drug concentrations during treatment. Therefore, these studies show that it is possible that, by increasing the difference or fluctuation between peak concentration and trough concentration of calcineurin-inhibitor drugs, a dosing regimen can be established that maximizes efficacy of the drug and minimizes the occurrence of nephrotoxicity. Maximizing the fluctuation between peak and trough concentrations may be accomplished by maximizing peak concentrations, minimizing trough concentrations, or both, or by increasing the time between doses, thereby allowing the trough concentration to reach a lower low point. In addition, Study B shows that nephrotoxicity can be predicted on the basis of fluctuation between peak and trough concentrations.

Based on these studies, an embodiment of the present invention is a method of therapeutic drug monitoring for calcineurin inhibitor therapy comprising taking a drug measurement at Cmax or C2, and taking an additional drug measurement at Cmin, a time at which the drug is in its lowest concentration in the body. Both of these measurements are considered in an embodiment of the therapeutic drug monitoring of the present invention. An additional embodiment of the present invention is a method of predicting a patient's tendency to develop toxicity associated with calcineurin-inhibition therapy based on analysis of data obtained by taking measurements at the two data points, Cmax and Cmin. Another embodiment of the present invention is a method of dosing calcineurin inhibitors where the calcineurin inhibitors are dosed in order to maximize Cmax, minimize Cmin, and maximize FLU, the fluctuation between Cmax and Cmin where this dosing regimen maximizes the efficacy and minimizes the toxicity associated with calcineurin inhibition therapy.

Study A explored the relationships between drug concentration and efficacy, by analyzing both renal transplant and psoriasis clinical data sets using ISA247, the cyclosporin A derivative described above. Pharmacokinetic and pharmacodynamic analyses were conducted on clinical trial results to compare the efficacy and toxicity of the experimental drug, ISA247, with CsA. In conducting pharmacokinetic and pharmacodynamic analyses, it became clear that efficacy of these calcineurin inhibitor drugs was correlated with peak concentrations, Cmax, and not, as had been anticipated, trough concentrations, C0. In addition, efficacy was not correlated with patient weight, patient clearance, or AUC suggesting that total systemic exposure is not a good indicator of drug efficacy. This suggests that calcineurin inhibitors demonstrate concentration dependent pharmacodynamics where the highest concentration correlates with maximum calcineurin inhibition and therefore the degree of immunosuppression. Calcineurin inhibition then trends towards normal towards the end of the dosing interval suggesting that sustained inhibition of calcineurin is not desirable.

An embodiment of this invention provides a method for maximizing the fluctuation between the peak concentration of cyclosporin and cyclosporin-related compounds, and calcineurin inhibitors as a class and the trough concentration of cyclosporin and cyclosporin-related compounds where maximizing the peak concentration of cyclosporin related compound ISA247 is associated with efficacy of the compound in inhibiting calcineurin activity and minimizing the trough concentration of cyclosporin related compound ISA247 minimizes toxicity and side-effects of the therapy, including renal toxicity.

Another embodiment of this invention relates to a method for predicting calcineurin toxicity based on a patient's peak-trough fluctuation. The less peak-trough fluctuation a patient exhibits, the greater the probability that the patient will suffer side effects associated with calcineurin inhibition therapy, specifically renal toxicity as measured by increasing levels of serum creatinine.

In another embodiment, this invention provides a once-daily dosing regimen for cyclosporin and cyclosporin-related compounds such as ISA247 (and possibly all calcineurin inhibitors) which maximizes peak concentration and maximizes efficacy, minimizes trough concentration and minimizes toxicity, and maximizes the peak-trough fluctuation, a predictor for cyclosporin-related renal toxicity.

Although this invention is exemplified with cyclosporin A and ISA247, a cyclosporin-related compound, the invention can be applied to therapy for other calcineurin inhibitor agents such as FK506, pimecrolimus and ascomycin, their analogs, derivatives, amides, esters, prodrugs and related compounds.

Study A: Pharmacokinetic and Pharmacodynamic Analysis of Clinical Trial Data

Study A compared the pharmacokinetics (PK) and pharmacodynamics (PD) of ISA247, a new-generation calcineurin inhibitor, with cyclosporine (CsA, Neoral®) using data from a phase II, randomized, multi-centre, open-label study in stable renal transplant patients. Stable renal transplant patients (≧6 months post-transplant) on an established dose of CsA were randomized to either continue CsA or switch to ISA247 for a 12 week period. ISA247 was estimated in pre-clinical and phase I studies to be at least 3-fold more potent that CsA and was dosed at one-third of the established CsA dose (mean study dose; 3.0±1.5 vs 1.2±0.6 mg/kg). At weeks 1, 6, and 12, serial whole blood samples were drawn and drug concentrations were determined. PK and PD were evaluated using standard non-compartmental analysis and a calcineurin inhibition assay. The calcineurin inhibition assay is modified from the method previously described by Fruman et al. (1992) and disclosed in U.S. Pat. No. 6,613,739 and U.S. Pat. No. 6,605,593. Whole blood lysates were evaluated for their ability to dephosphorylate a ³²P-labelled 19 amino acid peptide substrate in the presence of okadaic acid, a phosphatast type 1 and 2 inhibitor. Background phosphatase 2C activity (CsA and okadaic acid resistant activity) was determined and subtracted from each sample, with the assay performed in the presence and absence of excess added CsA. The remaining phosphatase activity was taken as calcineurin activity. Serum creatinine was measured from blood samples, as a measure of renal toxicity.

A direct PK-PD correlation was performed using a sigmoid Emax model. A total of 132 patients were recruited and randomized to ISA247 (n=65) and CsA (n=67), respectively. Patient demographics were similar between the groups with the exception of age (47.1±10.5 vs. 52.0±1 1.0 years, p<0.05). Time to maximum concentration (tₘₐₓ) and t_{1/2} were similar between the two drugs. For ISA247, the maximum concentration (Cmax) and the area under the concentration-time curve from 0-8 hours (AUC(0-8)) were approximately one third those of CsA.

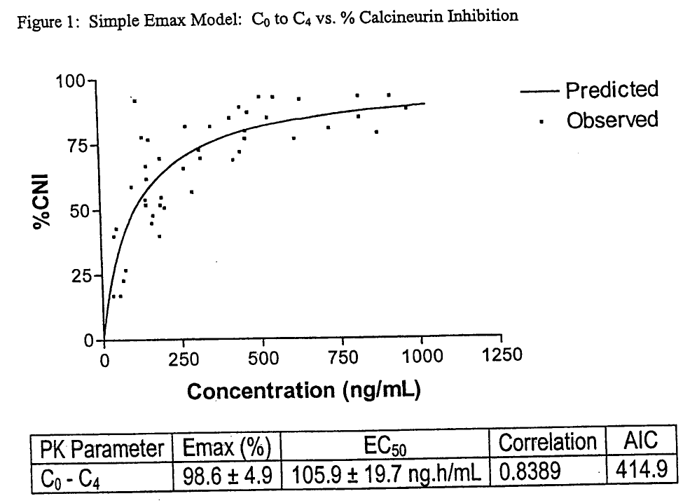

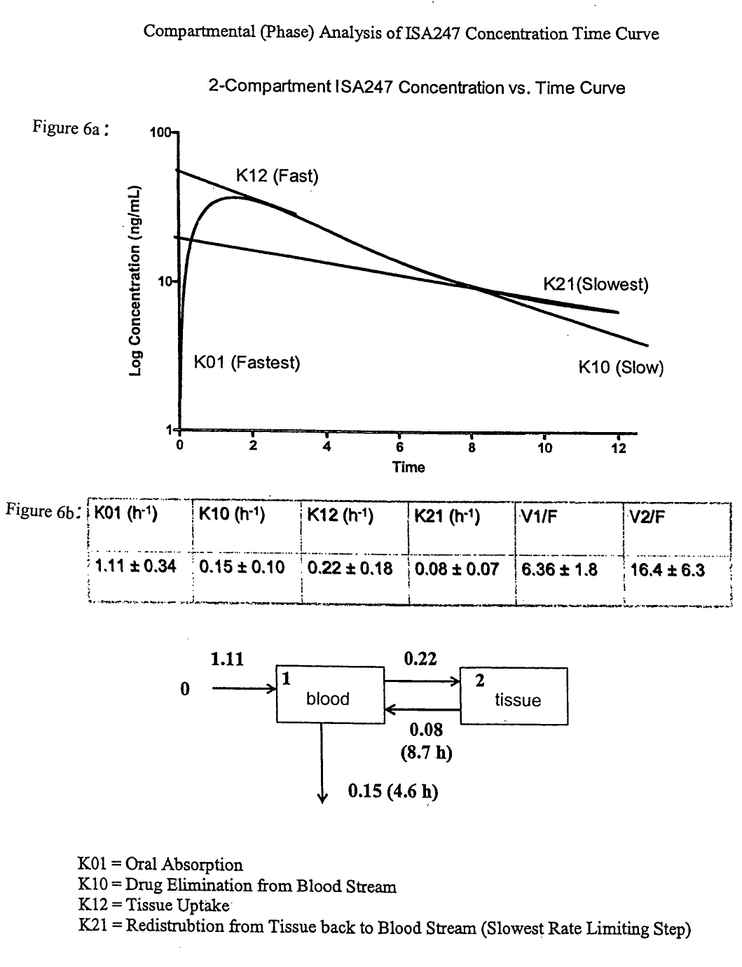

FIG. 1 illustrates that the effectiveness of ISA247, a cyclosporin related compound, as measured by percent calcineurin inhibition (% CNI), is dose-dependent. That is, % CNI increases with increasing drug concentration. FIG. 1 shows that there is a significant correlation (0.8339) between ISA247 whole blood concentration and percent calcineurin inhibition. According to FIG. 1, Emax, the maximum effective dose of ISA247 is 98.6 ng/mL±4.9 and the EC₅₀, the concentration at which % CNI is 50% is 105.9 ng.h/mL±19.7. The AIC is 414.9. AIC is the Akaike Information Criterion. It is a dimensionless “goodness of fit” parameter that is a modification of an ‘F-test.’ The lower the AIC number the better the fit. This relationship was constructed using concentrations from trough (C0) through hour 4 (C4). Any concentration in the absorptive phase of the drug (from time 0 to 4 hours, K01 in FIG. 6a) correlates well with calcineurin inhibition. Any concentration from the elimination phase (from 8 to 12 hours, for example, K10 in FIG. 6a) does not correlate well with calcineurin inhibition. See FIGS. 6a and 6b.

While % CNI is used in FIG. 1 to illustrate drug efficacy, there are other ways to measure transplant drug efficacy or calcineurin inhibitor drug efficacy. For example, in animal models, graft survival in transplantation is an indication of immunosuppressive drug efficacy. However, graft survival is an experimental model that takes a long time to measure and is complicated in that grafts may not survive for reasons other than the effective concentration of immunosuppressive agents. For example, prolonged cold ischemic time, donor/recipient mismatch or surgical technique may affect outcome of a graft. In human transplant patients, episodes of rejection may be indicators of failure of a calcineurin inhibitor. Other markers may be patient survival, in the case of transplant. For Psoriasis, a decrease in Psoriasis Area and Severity Index (PASI) score is an indication of effective treatment of psoriasis. A 75% reduction in PASI score is commonly held as a successful therapy.

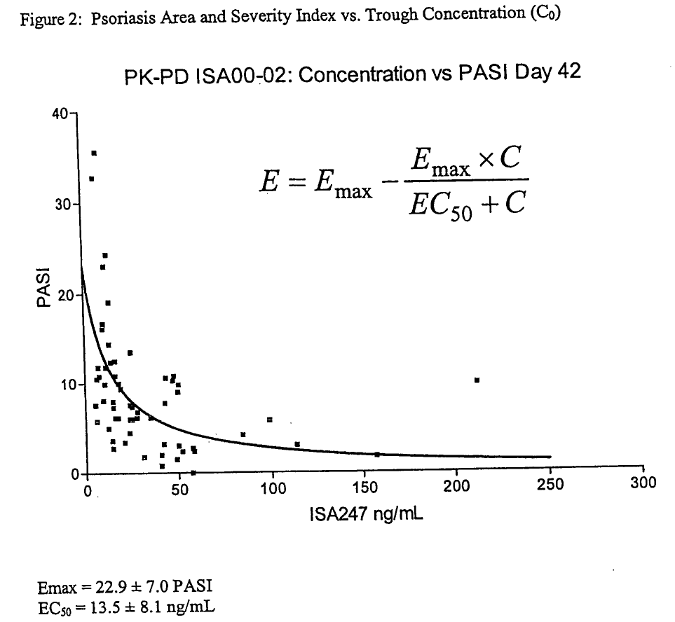

Instead of calcineurin inhibition, FIG. 2 shows the effect that ISA247 drug concentration has on reduction in PASI score, when given to psoriasis patients. Using PASI score as another indicator of drug efficacy, FIG. 2 illustrates that as drug concentration increases, the PASI score, an indicator of psoriasis severity, decreases. The curve illustrated in FIG. 2 is represented by Formula 1:

$$Formula 1 \text{:}$$

$$E = {E}_{max} - \frac{{E}_{max} \times C}{{EC}_{50} + C}$$

Where E is the effective dose, Emax is the maximum possible mean PASI, C is the concentration of drug, and EC₅₀ is the concentration at which the reduction of PASI score is at 50%. EC50 is an indication of the potency of the drug. Formula 1 is a modification of the Hill equation used to describe drug receptor interaction or drug-effect relationships. FIG. 2 illustrates that as concentration goes up, PASI goes down. In this study, Eₘₐₓ is 22.9±7 PAS₁ and EC₅₀ is 13.5±8.1 ng/ml ISA247 where EC50 is the effective concentration required to achieve a 50% reduction in PASI. In this study, trough (C0) concentrations were the sole pharmacokinetic measurement. Therefore the EC50 represents the trough concentration required to achieve a 50% reduction in PASI. FIG. 2 illustrates the relationship between trough drug concentration (C₀) and PASI score on day 42 of treatment where E equals effect, in this case measured by PASI score.

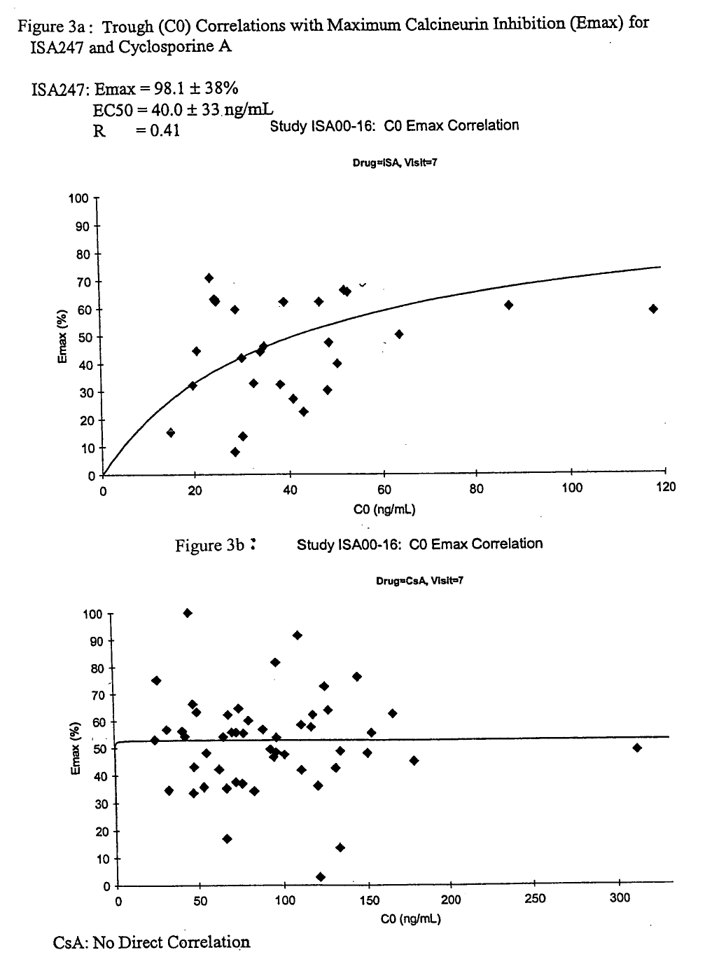

FIGS. 3A and 3B plot trough concentration (C0) of calcineurin inhibitor drugs ISA247 and cyclosporin A (Neoral®) versus Eₘₐₓ, the maximum effectiveness, calculated as percent effectiveness. FIG. 3A shows the correlation between ISA trough concentration (C0) and Emax measured at day 7 of the 12 week clinical trial described above. As illustrated in FIG. 3A, the correlation or relationship is very weak (R=0.41, or a 41% correlation with a very high standard error of ±33) between Emax and trough concentration (C0) for ISA247-treated patients. For patients treated with cyclosporin A (CsA, Neoral®), there is no measurable correlation between Emax and trough concentration (C0) measured at day 7 of the same clinical trial.

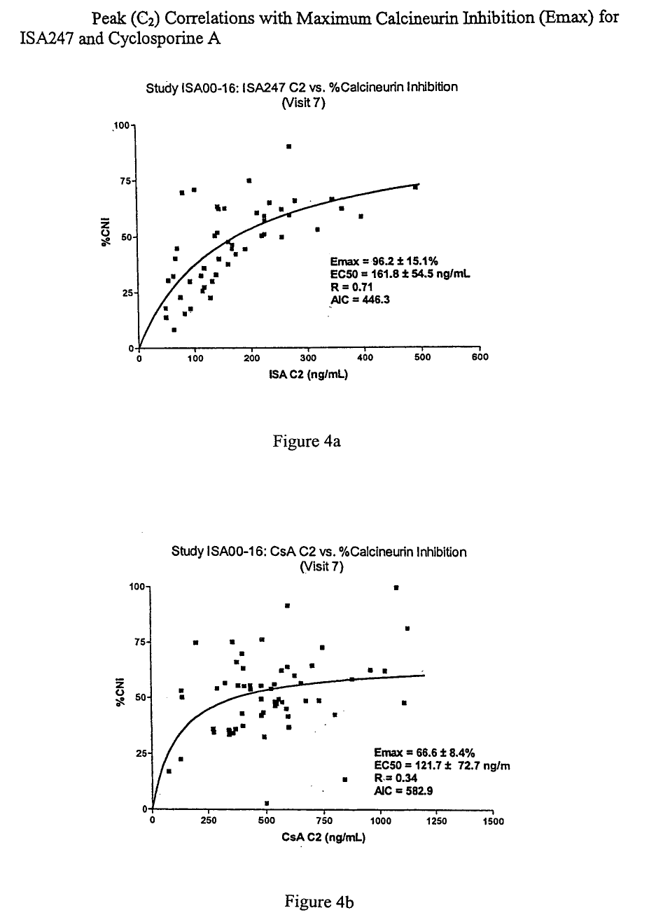

FIGS. 4A and 4B show the correlation between % CNI and peak concentrations of ISA and CsA (Neoral®) (measurements taken at 2 hours, C2, are presumed to be the peak blood drug concentration) in patients enrolled in the clinical trial described above. FIG. 4A shows that there is a strong correlation between peak ISA concentration and percent calcineurin inhibition. In FIG. 4A, Emax (or C2) is calculated at 96.2±15.1%, EC50 is calculated at 161.8+54.5 ng/mL, and the correlation between peak ISA247 concentration and maximum calcineurin inhibition (Emax) is 71% (R=0.71).

In FIG. 4B, Emax (or C2) for CsA (Neoral®) is calculated at 66.6+8.4%, EC50 is calculated at 121.7+72.7 ng/mL, and the correlation between peak CsA concentration and maximum calcineurin inhibition (Emax) is 34%. While the correlation is stronger for ISA than for CsA (0.71 and 0.34 respectively), the correlation between peak concentration and Emax for both ISA and CsA is greater than the correlation between trough concentration and Emax (see FIGS. 3A and 3B). Therefore, it is not the trough concentration of calcineurin inhibitor drugs that determines efficacy, as measured by % calcineurin inhibition (see FIGS. 3A and 3B). It is the peak concentration of calcineurin inhibitor drugs that determines efficacy, as measured by % calcineurin inhibition (see FIGS. 4A and 4B).

Study B: Discriminant Analysis of Pharmacokinetic Data

Study B is a Discriminant Analysis perfomed on the data obtained in the same Phase II clinical trial described above, to determine which variable(s) discriminate(s) between the group of patients who experienced renal toxicity as a result of treatment with ISA247, as measured by increased serum creatinine, and patients who did not experience renal toxicity. This discriminant analysis was performed using SPSS for Windows version 10.1 software available from SPSS Inc.

As will be clear from someone of ordinary skill in the art, discriminant analysis is a statistical method for determining which variables discriminate between two or more naturally occurring groups. Here, discriminant analysis was used to discriminate between the Normal Renal Function Group, (NRFG) and the Renal Toxicity Group (RTG).

TABLE 1
Discriminant Parameters

| | | | Valid N (listwise) | | |
|---|---|---|---|---|---|
| | | | Standard | | |
| Group | | Mean | Deviation | Unweighted | Weighted |
| NRFG | C8 | 36.699 | 26.4878 | 121 | 121.0000 |
| | Cmin | 30.255 | 19.5701 | 121 | 121.0000 |
| | Fluctuate | 459.672 | 186.5907 | 121 | 121.0000 |
| RTG | C8 | 54.327 | 30.9933 | 26 | 26.000 |
| | Cmin | 39.358 | 20.9217 | 26 | 26.000 |
| | Fluctuate | 312.053 | 135.0751 | 26 | 26.000 |
| Total | C8 | 398.817 | 28.0480 | 147 | 147.000 |
| | Cmin | 31.865 | 20.0471 | 147 | 147.000 |
| | Fluctuate | 433.562 | 186.9076 | 147 | 147.000 |

NRFG = Normal Renal Function Group
RTG = Renal Toxicity Group (≧ 15% Rise in Serum Creatinine from Baseline)
C8 = Eight Hour ISA247 Whole Blood Concentration
Cmin = Minimum ISA247 Whole Blood Concentration Measured
Fluctuate = % Fluctuation ([Cmax − Cmin]/Cavg * 100)

Table 1 shows that for Group NRFG, patients (n=121) who exhibited Normal Renal Function during the course of the clinical trial described above, the mean Fluctuation between peak drug concentration (a measurement taken 8 hours after dosing with ISA247) and trough concentration (Cmin) was 459.672 ng/mL with a standard deviation of 186.591. A measurement taken at 8 hours was used as peak concentration in the absence of a C2 measurement in this study. For Group RTG, patients (n=26) who exhibited renal toxicity≧15% rise in serum creatinine from baseline, the mean fluctuation between peak drug concentration (a measurement taken 8 hours after dosing with ISA247) and trough concentration (Cmin) was 312.013 ng/mL with a standard deviation of 135.0751. Cmin measurement could be a measurement taken at C0 or at C8, whichever measurement gave the lower measurement. Those of ordinary skill in the art will recognize that clinically, definitions of renal toxicity may be significantly higher increases of serum creatinine from baseline. Clinical diagnosis of renal toxicity may require an increase of, for example, 50% over baseline. For the purposes of this data analysis, however, an increase of 15% over baseline was used.

According to this analysis, the fluctuation between the peak concentration of the drug and the trough concentration of the drug is a key factor to consider when predicting toxicity as a result of treatment with the calcineurin inhibitor ISA247.

Surprisingly, Cmax and Cavg (the average concentration of drug) did not have any predictive value. That is, Cmax and Cavg, according to this discriminant analysis, were not factors that were important to consider in predicting whether a patient will suffer from renal toxicity, as defined as a 15% increase in serum creatinine over baseline when dosed with the calcineurin inhibitor ISA247. Even more surprisingly, AUC did not have a predictive value for renal toxicity in this analysis. AUC, as a measure of the cumulative concentration of drug, would be expected to be a predictor of toxic effect associated with total systemic drug exposure.

AUC, a measurement which has been so important in determining appropriate dosing levels for CsA and calcineurin inhibitors, and a measurement that has been approximated by measurements of C2 drug concentrations, does not discriminate between peak levels and trough levels, but effectively averages these measurements over a dosing period. Therefore, according to this analysis, AUC is not a good benchmark to use in determining either calcineurin inhibitor efficacy or to predict toxicity, because AUC as a measurement ignores both peak and trough concentrations and therefore does not contain the most important measurements in predicting efficacy or toxicity of calcineurin inhibition.

TABLE 2
Discriminant Standardized Canonical Correlation Coefficients

| | Function Group RTG |
|---|---|
| C8 | −1.205 |
| Cmin | .957 |
| Fluctuate | .754 |

Table 2 describes the standardized canonical correlation coefficients for Discriminant analysis. The larger the correlation coefficient, the more important is the factor in discrimination of the groups. The data points presented in Table 2, taken in relation to each other, were the largest coefficients and therefore the most important factors as determined by this analysis.

TABLE 3
Statistical Significance for Discrimant Analysis

| Test of | | | | |
|---|---|---|---|---|
| Function(s) | Wilks' Lambda | Chi-square | Df | Significance |
| 1 | .880 | 18.293 | 3 | .000 |

Table 3 describes the stastical significance for discriminant analysis. It is important in discriminant analysis to ensure that selected factors are statistically significant at a level of α≦0.5. Wilks' Lambda and Chi squared are commonly used in this assessment.

TABLE 4
Group Classification

| | | | Predicted Group Membership | |
|---|---|---|---|---|
| | Actual Group | | Correctly | Incorrectly |
| GROUP | Membership | | Placed | Placed |
| NRF | Number | 121 | 118 | 3 |
| RT | | 26 | 23 | 3 |
| Ungrouped | | | 4 | 0 |
| NRF (%) | Percent | 82.3% | 97.5% | 2.5% |
| RT (%) | | 17.7% | 88.5% | 11.5% |
| Ungrouped (%) | | 100% | 100.0% | .0% |

Table 4 shows the actual and predicted membership in the two groups, normal renal function (NRF) group and the renal toxicity (RT) group. Analysis of the results of the clinical trial indicated that, of the 147 patients participating in the trial, and being dosed with ISA247, 121 patients exhibited normal renal function and 26 patients exhibited renal toxicity. Table 4 illustrates the number of patients who were correctly classified as NRF, non renal failure patients, and the number of patients classified as RF, renal failure patients, compared to actual groups. NRF patients were correctly classified using this analysis 97.5% of the time. 118 out of 121 patients in the NRF group were correctly placed in the NRF class using this predictive method. RF patients were correctly classified 88.5% of the time. 23 of 26 patients were correctly placed in the RF class using this predictive method.

Considering the fluctuation between peak and trough drug levels, this discriminant analysis method was able to predict that 118 patients would be in the normal renal function group and 23 patients would be in the renal toxicity group. These predictions were correct, for placement in the normal renal function group, 97.5% of the time (2.5% of the time this placement was in error), and for placement in the RT group 88.5% of the time (11.5% of the time this placement was in error). By the discriminant analysis presented here.

TABLE 5
Descriptive Statistics Comparing
Normal vs. Renal Toxicity Groups

| | | | | Std. | Std. Error |
|---|---|---|---|---|---|
| | Group | N | Mean | Deviation | Mean |
| Cmax | NRF | 123 | 187.67 | 89.30 | 7.83 |
| Cmax | RT | 26 | 159.59 | 87.7 | 20.67 |
| Cavg | NRF | 123 | 37.05 | 19.67 | 1.74 |
| Cavg | RT | 26 | 42.3 | 23.47 | 5.53 |
| C8 | NRF | 123 | 37.311 | 28.1350 | 2.5368 |
| C8 | RT | 26 | 54.327 | 30.9933 | 6.0783 |
| Cmin | NRF | 123 | 29.957 | 19.5674 | 1.7643 |
| Cmin | RT | 26 | 39.358 | 20.9217 | 4.1031 |
| FLUCTUATE | NRF | 121 | 459.672 | 186.5907 | 16.9628 |
| FLUCTUATE | RT | 26 | 312.053 | 135.0751 | 26.4904 |

Table 5 shows arithmetic means for blood concentration measurements taken from patients in the Normal Renal Function Group (NRF) and the Renal Toxicity group (RT) Group at Cmax, Cavg, C8 (which was usually Cmin) and Cmin. Differences between the NRF and RT Cmax and Cavg measurements were not statistically significant. However, as shown in Table 6, differences between the NRF and RT C8 and Cmin measurements were statistically significant. Mean C8 (measurement taken at 8 hours) for the NRF group (n=123) was 37.311 ng/mL. Cmin (the lowest of either C0 or C8) measurements were lower for the NRF (mean=29.957 ng/mL) than for the RT (mean=54.327 ng/mL).

Percent fluctuation (FLU) was calculated as shown in Formula (2):

$$Formula ( 2 ) : \\ FLU = \frac{{C}_{max} - {C}_{min}}{{C}_{avg}} \times 100$$

Importantly, mean fluctuation between Cmax and Cmin for the RT group was significantly lower (312.053%) compared to the mean fluctuation between Cmax and Cmin or the NRF group (459.672%).

Table 5 shows that patients in the RT group, patients who experienced renal toxicity as measured by elevated serum creatinine, had a higher Cmin than patients in the NRF group. Therefore, lower Cmin, or lower trough levels of drug, are associated with lower toxicity of ISA247, a calcineurin inhibitor.

Table 5 also shows that the fluctuation between Cmax and Cmin for patients dosed with ISA247 was much higher for patients in the NRF group than the fluctuation between Cmax and Cmin for patients in the RT group. Therefore, lower fluctuation between Cmax and Cmin, or peak and trough levels of ISA247, is predictive for membership in the RT group. Fluctuation between peak and trough levels of drug is predictive for toxicity for calcineurin inhibitor drugs.

TABLE 6
t-Test for Independent Samples Comparing Normal vs. Renal Toxicity Groups

| | | Levene's | | t-test for Equality of Means | | | | | | |
|---|---|---|---|---|---|---|---|---|---|---|
| | | Test for | | | | | | | 95% Confidence | |
| | | Equality of | | | | | | | Interval of the | |
| | | Variances | | | | Sig. | Mean | Std Error | Difference | |
| | | F | Sig | T | Df | (2-tailed) | Difference | Difference | Lower | Upper |
| C8 | EVA | .939 | .334 | −2.752 | 147 | .007 | −17.015 | 6.1822 | −29.2327 | −4.7976 |
| | EVNA | | | 2.583 | 34.255 | .014 | −17.015 | 6.5864 | 30.3967 | −3.6336 |
| Cmin | EVA | .136 | .713 | 2.199 | 147 | .029 | −9.401 | 4.2748 | 17.8489 | −.9531 |
| | EVNA | | | 2.105 | 34.856 | .043 | −9.401 | 4.4663 | 18.4695 | −.3325 |
| FLU | EVA | 4.005 | .047 | 3.820 | 145 | .000 | 147.619 | 38.6435 | 71.2417 | 223.9963 |
| | EVNA | | | 4.693 | 48.023 | .000 | 147.609 | 31.4560 | 84.3733 | 210.8647 |

Table 6 shows t-Test analysis for C8, Cmin and fluctuation. The table first tested the equality of the variance using a Levene's test. The Levene's test indicated equal variances should be used for C8 and Cmin, while unequal variances must be used for fluctuation, FLU. For all parameters tested, the difference between the RF group and the NRF group were statistically significant. Therefore the renal failure group had less peak-trough fluctuation and higher trough and C8 levels than the NRF group. This suggests a critical trough concentration is necessary to minimize nephrotoxicity caused by calcineurin inhibitors. Patients with greater peak-trough fluctuation may achieve this critical trough concentration after twice daily dosing. Patients who cannot achieve this critical trough concentration or peak-trough fluctuation are at risk for developing nephrotoxicity. A dosing regimen which maximizes the peak-trough fluctuation (FLU) will minimize toxic side effects associated with the use of calcineurin inhibitor drugs. Once daily dosing may ensure the majority of patients will have adequate peak-trough fluctuation resulting in trough levels below the nephrotoxic threshold. The risk of reduced peak-trough fluctuation and trough levels above the nephrotoxic level may be increased by twice daily dosing.

Based on the above analysis, it is possible to predict the nephrotoxicity exhibited by patients dosed with calcineurin inhibitors by measuring (1) their trough drug concentration; and (2) their peak-trough fluctuation. Peak-trough fluctuation should be determined by intrinsic drug clearance. Unfortunately, in the clinical environment, intrinsic clearance is difficult to measure as it is confounded by oral bioavailability of a drug. Peak concentration (C2) and trough concentration (C0) are easily measured, and can be used to predict a patient's risk for the development of nephrotoxicity. Using these t-Test results, it can be seen that there is a statistically significant difference between the RT group and the NRF group based on these parameters.

There is a dose level that you need to be below at one point in order to avoid toxicity. C8 of 37.311 ng/mL, Cmin of 29.957 ng/mL and low fluctuation (less than 459.62) are correlated with toxicity. Cmin is defined as the lowest (minimum) concentration measured during the dosing interval (it was either a C0 or C8 in this study). Cmin can occur at anytime but is more likely to occur just prior to receiving a new dose.

FIGS. 6a and 6b show fluctuation in concentration of ISA247 in whole blood over a period of from 0 to 12 hours, after an initial dose at time 0. FIG. 6a illustrates the multiple phases of the drug concentrations that can be compartmentalized into phases. FIG. 6b shows a kinetic analysis of the concentration curve shown in FIG. 6a. FIG. 6b illustrates two compartments. Compartment 1 is the blood or the bloodstream, and compartment 2 is tissue. This kinetic model shows rapid uptake in to the tissue followed by slower elimination from the tissue. In this model, K01 is oral absorption of the drug which occurs rapidly, as shown in FIG. 6a as the rapid initial upward rise in concentration. This rapid first step is followed by distribution of drug from the blood (compartment 1) into tissue (compartment 2), or Tissue Uptake. This step, K12, is relatively fast. This step is followed by K21, the redistribution of drug from the tissue back to the blood stream. According to this model, this step is the slowest and rate limiting step. Finally, the drug is eliminated from the blood stream in step K10. This step is relatively slow.

In accordance with this model, the higher the peak concentration, Cmax, the more of a driving force is present to drive the drug into the tissues. Without wishing to be bound to a specific theory, this model may explain the correlation that was seen between peak drug concentration and drug efficacy in FIGS. 1, 2 and 4. Pharmacokinetics of ISA247 were determined fitting whole blood concentration data to a 2-compartment model using the nonlinear regression software package, WinNonlin Professional v. 4.1 (Pharsight, Mountain View Calif.). The higher the peak, the more drug will be present in the tissues. Coupled with slow elimination from tissues to the blood (K21) and from blood to elimination (K10), a high peak concentration may cause drug to reside in the tissues where it is most effective to create a more effective dose of drug. This may be especially true in the treatment of a condition such as psoriasis, where the drug must be in the skin to be effective. While increasing the peak concentration may act to drive drug into the tissues, decreasing the trough concentration may act to pull drug out of the tissues. This may decrease toxicity by allowing the tissues that exhibit toxic effects such as the kidneys to recover from the nephrotoxic effects of the drug that occur at higher drug concentration.

### EXAMPLE 1

### Once Daily Dose Optimizes Efficacy and Minimizes Toxicity

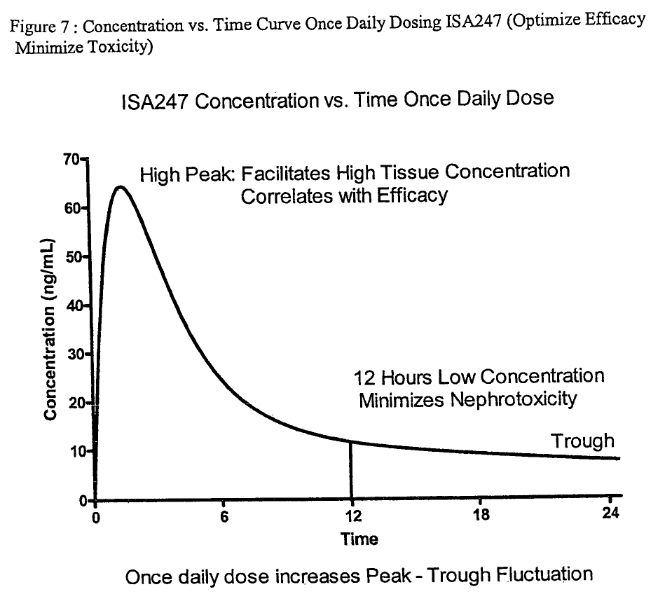

FIG. 7 illustrates a theoretical concentration time curve for dosing of ISA247 which optimizes efficacy and minimizes toxicity. In FIG. 7, the peak concentration is maximized at approximately 65 ng/mL. The concentration is then allowed to drop below a threshold level of 30 ng/mL (see Table 5) to minimize toxicity. The fluctuation between peak concentration and trough concentration is maximized to decrease the patient's risk of developing nephrotoxicity. This dosing strategy allows for a 12 hour period in which concentrations are below the critical nephrotoxic threshold. While the mechanism is not known, this may allow for adaptation in the kidney, or may allow the tissue to repair itself or allow for thorough tissue perfusion for a period of time. Depending on the patient, doses at between 24 and 48 hours may also be optimal.

### EXAMPLE 2

### Sustained Release Dosing

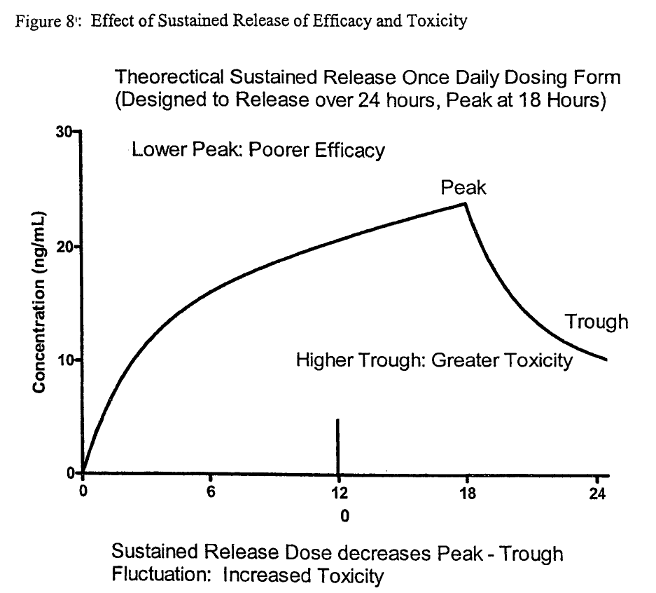

As a comparison, FIG. 8 illustrates a typical sustained-release concentration curve. In this dosing regimen, a target concentration is identified and a dosing regimen is established to maximize the time spent at or near the target concentration. This type of sustained-release dosing decreases peak-trough fluctuation, minimizes peak concentration and increases trough concentration. While this may be preferable for some medications, in the case of calcineurin inhibitors such as ISA247, and also CsA and FK506, this dosing regimen would not maximize efficacy and would increase toxic side effects of the drug.

All of the cited references are incorporated herein by reference in their entirety. While the above-described embodiments particularly show and describe the invention, it will be understood by those skilled in the art that various changes in form and details may be made therein without departing from the spirit and scope of the invention as defined by the appended and finally allowed claims.

1. A method for administering a calcineurin inhibitor to a patient in need of calcineurin inhibition therapy which optimizes efficacy of the calcineurin inhibitor and minimizes calcineurin inhibitor-related toxicity comprising maximizing the fluctuation between a peak calcineurin inhibitor concentration and a trough calcineurin inhibitor concentration.

2. The method of claim 1 wherein the calcineurin inhibitor is selected from the group consisting of cyclosporine A, cyclosporine A derivatives, ISA247, FK506, pimecrolimus and ascomycin.

3. The method of claim 1 wherein the calcineurin inhibitor is administered once daily.

4. The method of claim 1 wherein the trough concentration is minimized.

5. The method of claim 1 wherein the time at trough is maximized.

6. The method of claim 1 wherein the calcineurin inhibitor is ISA247.

7. The method of claim 3 wherein the calcineurin inhibitor is ISA247.

8. A method for administering a calcineurin inhibitor comprising administering the calcineurin inhibitor once daily wherein the once daily dose maximizes peak concentration of the calcineurin inhibitor and minimizes trough concentration of the calcineurin inhibitor.

9. The method of claim 8 wherein the once daily dose method maximizes peak-trough fluctuation.

10. The method of claim 8 wherein the calcineurin inhibitor is selected from the group consisting of cyclosporine A, cyclosporine A derivatives, ISA247 and FK506.

11. A method for monitoring a patient receiving calcineurin inhibitor therapy comprising:
(1) measuring the patient's peak concentration of a calcineurin inhibitor; and,
(2) measuring the patient's trough concentration of a calcineurin inhibitor.

12. The method of claim 11 wherein the calcineurin inhibitor is selected from the group consisting of cyclosporine A, cyclosporine A derivatives, ISA247 and FK506.

13. The method of claim 11 further comprising:
(1) calculating a peak-trough fluctuation; and,
(2) using the calculated peak-trough fluctuation as a marker to monitor for the development of calcineurin-inhibitor therapy-related toxicity in the patient wherein a smaller peak-trough fluctuation indicates a greater probability that the patient will suffer calcineurin inhibition therapy-related toxicity.

14. The method of claim 13 wherein the calcineurin inhibitor is selected from the group consisting of cyclosporine A, cyclosporine A derivatives, ISA247, FK506, pimecrolimus and ascomycin.

15. The method of claim 13 wherein when the calculated peak-trough fluctuation is less than 350%, toxicity is predicted.

16. A method for monitoring a patient receiving calcineurin inhibition therapy to predict calcineurin inhibition therapy-related toxicity in a patient comprising:
(a) measuring the patient's peak concentration of a calcineurin inhibitor;
(b) measuring the patient's trough concentration of a calcineurin inhibitor.

17. The method of claim 16 wherein the calcineurin inhibitor is selected from the group consisting of cyclosporine A, cyclosporine A derivatives, ISA247, FK506, pimecrolimus and ascomycin.

18. A method for predicting calcineurin inhibition therapy-related toxicity in a patient comprising:
(1) measuring the patient's peak concentration of a calcineurin inhibitor;
(2) measuring the patient's trough concentration of a calcineurin inhibitor;
(3) calculating a peak-trough fluctuation; and,
(4) using the calculated peak-trough fluctuation to predict toxicity in the patient wherein a smaller peak-trough fluctuation indicates a greater probability that the patient will suffer calcineurin inhibition therapy-related toxicity.

19. The method of claim 18 wherein when the calculated peak-trough fluctuation is below 350%, toxicity is predicted.

20. The method of claim 20 wherein the calcineurin inhibitor is selected from the group consisting of cyclosporine A, cyclosporine A derivatives, ISA247, FK506, pimecrolimus and ascomycin.

21. A method for administering a calcineurin inhibitor to a patient in need of calcineurin inhibition therapy which optimizes efficacy of the calcineurin inhibitor and minimizes calcineurin inhibitor-related toxicity comprising maximizing the fluctuation between peak calcineurin inhibition and trough calcineurin inhibition.

22. The method of claim 21 wherein the peak and trough calcineurin inhibition are measured after administration of a calcineurin inhibitor selected from the group consisting of cyclosporine A, cyclosporine A derivatives, ISA247, FK506, pimecrolimus and ascomycin.

23. The method of claim 22 wherein the calcineurin inhibitor is administered once daily.

24. The method of claim 1 wherein the time at trough calcineurin inhibition is maximized.

25. The method of claim 22 wherein the calcineurin inhibitor is ISA247.

26. The method of claim 23 wherein the calcineurin inhibitor is ISA247.

27. A method for monitoring a patient receiving calcineurin inhibitor therapy comprising:
(1) measuring the patient's peak calcineurin inhibition; and,
(2) measuring the patient's trough calcineurin inhibition.

28. The method of claim 27 wherein the calcineurin inhibitor is selected from the group consisting of cyclosporine A, cyclosporine A derivatives, ISA247, FK506, pimecrolimus and ascomycin.

29. The method of claim 27 further comprising:
(a) calculating a peak-trough calcineurin inhibition fluctuation; and,
(b) using the calculated peak-trough calcineurin inhibition fluctuation as a marker to monitor for the development of calcineurin-inhibitor therapy-related toxicity in the patient wherein a smaller peak-trough calcineurin inhibition fluctuation indicates a greater probability that the patient will suffer calcineurin inhibition therapy-related toxicity.

30. The method of claim 29 wherein the calcineurin inhibitor is selected from the group consisting of cyclosporine A, cyclosporine A derivatives, ISA247, FK506, pimecrolimus and ascomycin.
